(19) United States
(12) Patent Application Publication
(10) Pub. No.: US 2003/0224459 A1
(43) Pub. Date: Dec. 4, 2003

(54) Protein detection method

(75) Inventors: Fumiko Yasukawa, Tokyo (JP); Takeshi Sasayama, Tokyo (JP); Shingo Ueno, Tokyo (JP)

Correspondence Address: Reed Smith Hazel & Thomas LLP, Suite 1400, 3110 Fairview Park Drive, Falls Church, VA, 22042-4503, US

(73) Assignee: Hitachi Software Engineering Co., Ltd.

(21) Appl. No.: 10/442,231

(22) Filed: May 21, 2003

(30) Foreign Application Priority Data
Jun. 4, 2002 (JP) 2002-162641

(51) Int. Cl. G01N 33/53; G01N 33/542

(52) U.S. Cl. 435/7.9

(57) ABSTRACT

A means of simultaneously detecting the characteristics of a plurality of proteins is provided. The protein detection method is a method of simultaneously detecting a plurality of proteins contained in a sample and comprises the steps of: arranging, on a substrate, a plurality of substances to be arranged, which form specific complexes with analytes; forming a plurality of first specific conjugates by allowing a specific binding reaction to occur on the above substrate between the analytes contained in the sample and the above plurality of arranged substances; forming a plurality of second specific complexes by allowing a specific binding reaction to occur between substances, which form specific complexes with the analytes and are labeled with a fluorescence reagent or an RI reagent, and the above plurality of first specific conjugates; and identifying the analytes contained in the sample by detecting the labels contained in the above plurality of second specific complexes.

### BACKGROUND OF THE INVENTION

1. Technical Field

The present invention relates to a method of detecting protein, which is useful in investigating and testing the identification, modification, expression analysis, interaction, functional analysis and quantitative determination of proteins in the field of protein analysis technology.

2. Background Art

Biological reaction is basically composed of intermolecular action and molecular recognition. In particular, proteins play a central role in the expression of physiological functions. Recently, human gene analysis has advanced to reveal that genes having unknown functions are present at approximately 40% of all the identified genes. Such proteins having unknown functions are increasingly being analyzed.

Currently, mainstream methods employed in the identification and quantitative determination of protein include a method using two-dimensional electrophoresis and mass spectrometry, and a method using liquid chromatography and mass spectrometry. Further, the detection of interaction and the identification of proteins have now begun to be performed using an antibody chip, which is an application of a DNA chip and provided with a number of antibodies spotted on the plane surface.

However, conventional methods using electrophoresis have problems in terms of resolution and detection sensitivity. To simultaneously analyze molecular reactions among molecules in vivo, a method, which causes a competitive binding reaction such as an antigen-antibody reaction of a number of proteins on a substrate, is effective. However, even an antibody chip, to which the method is applicable, requires protein to be previously fluorescent-labeled, so that the study of the characteristics of more proteins cannot be realized. An object of the present invention is to provide a method of simultaneously detecting reactivities of a plurality of proteins without previously labeling sample proteins.

### SUMMARY OF THE INVENTION

To achieve the above object, the protein detection method of the present invention is a method of simultaneously detecting a plurality of proteins contained in a sample and comprises the steps of: arranging, on a substrate, a plurality of substances to be arranged, which form specific complexes with analytes; forming a plurality of first specific conjugates by allowing a specific binding reaction to occur on the above substrate between the analytes contained in the above sample and the above plurality of arranged substances; forming a plurality of second specific complexes by allowing a specific binding reaction to occur between substances, which form specific complexes with the analytes and are labeled with a fluorescence reagent or an RI reagent, and the above plurality of first specific conjugates; and identifying the analytes contained in the sample by detecting the labels contained in the above plurality of second specific complexes.

Particularly, the above binding reaction is preferably an antigen-antibody reaction. In this case, the protein detection method of the present invention is a method of detecting a plurality of analytes that are antigens and/or antibodies contained in a sample, and comprises the steps of: arranging, on a substrate, a plurality of substances to be arranged, which are antibodies and/or antigens and form specific complexes with the above plurality of analytes; forming a plurality of first antigen-antibody complexes by allowing a specific antigen-antibody reaction to occur on the above substrate between the analytes contained in the sample and the above plurality of arranged substances; forming a plurality of second antigen-antibody complexes by allowing a specific antigen-antibody reaction to occur between the antibodies and/or antigens, which form specific complexes with the analytes and are labeled with a fluorescence reagent or an RI reagent, and the above plurality of first antigen-antibody complexes; and identifying the analytes contained in the sample by detecting the labeled substances contained in the above second antigen-antibody complexes.

Further, the above two binding reactions are not limited to the antigen-antibody reaction, and either or both reactions may be binding reactions other than the antigen-antibody reaction. In this case, the protein detection method of the present invention is characterized in that: the binding reaction, which occurs during the step of forming a plurality of first specific conjugates by allowing a specific binding reaction to occur on the above substrate between analytes contained in the sample and the above plurality of arranged substances, is selected from a specific antigen-antibody reaction between antibodies and antigens, binding reaction with peptides, interaction with proteins, enzyme reaction, and hybridization reaction with DNA; and the binding reaction, which occurs during the step of forming a plurality of second specific complexes by allowing a specific binding reaction to occur between the above plurality of first specific conjugates and the substances labeled with a fluorescence reagent or an RI reagent, is selected from a specific antigen-antibody reaction between antibodies and antigens, and binding reaction with peptides, interaction with proteins, enzyme reaction, and hybridization reaction with DNA.

In the present invention, the above substrate is preferably a plane basal plate or a bead, and the quality of the material is not limited.

Furthermore, the method of the present invention preferably includes a step of washing samples containing analytes that remain unreacted after the binding reaction, which is between the step of forming a plurality of first specific conjugates by allowing the specific binding reaction to occur on the above substrate between the analytes contained in the above sample and the above plurality of arranged substances, and the step of forming a plurality of second specific complexes by allowing a specific binding reaction to occur between the above plurality of first specific conjugates and the substances labeled with a fluorescence reagent or an RI reagent.

Similarly, the method of the present invention preferably includes a step of removing substances that have been labeled with a fluorescence reagent or an RI reagent and remain unbound after the binding reaction, which is between the step of forming a plurality of second specific complexes by allowing the specific binding reaction to occur between the above plurality of first specific conjugates and substances labeled with the fluorescence reagent or an RI reagent, and the step of identifying analytes contained in a sample by detecting the labeled substances contained in the above plurality of second specific complexes.

Further, the method of the present invention preferably includes a step of masking portions unspotted with substances arranged on a substrate, which is between the step of arranging, on the above substrate, a plurality of substances to be arranged, which form specific complexes with analytes, and the step of forming a plurality of first specific conjugates by allowing a specific binding reaction to occur on the above substrate between the analytes contained in the above sample and the above plurality of arranged substances.

As described above, according to the present invention, protein can be detected by labeling substances, which form specific complexes with analytes, without labeling the sample protein. Moreover, arranging a plurality of substances on a plate or beads makes it possible to simultaneously detect the reactivities of a plurality of proteins.

In addition, examples of “the substrate” in the present invention include a plate such as a slide glass, plastic substrate, or membrane, or is a bead such as a plastic bead or glass bead, and also include a substrate with its surface coated with an adjuvant that is for binding a greater number of proteins. “Analyte” is the generic name of any substance to be analyzed, and is a protein including an antibody, antigen, enzyme and the like. “Substances forming specific complexes with analytes” include protein such as an antibody, peptide or DNA, which is a recognition site, biopolymer such as a polynucleotide, and compound such as a phosphate group. “A specific binding reaction with analytes” represents an in vivo reaction such as a specific antigen-antibody reaction between antibodies and antigens, binding reaction with peptides, interaction with proteins, enzyme reaction, or hybridization reaction with DNA. “Substances labeled with a fluorescence reagent or an RI reagent” include substances that are not labeled before the binding reaction but labeled after the binding reaction, and substances labeled by enzyme reaction.

Further, a number of spots containing analytes are arranged on a substrate. The spots can be formed by a spotter system for preparing various arrays.

### BRIEF DESCRIPTION OF THE DRAWINGS

FIG. 1 shows a flowchart of the embodiment of the present invention.

FIG. 2 shows reaction models of the embodiment of the present invention.

### DETAILED DESCRIPTION OF PREFERRED EMBODIMENTS

The invention will be hereafter described by way of embodiments with reference made to the drawings.

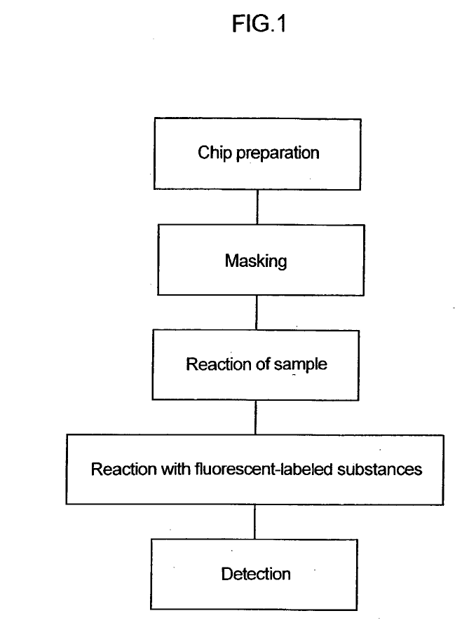

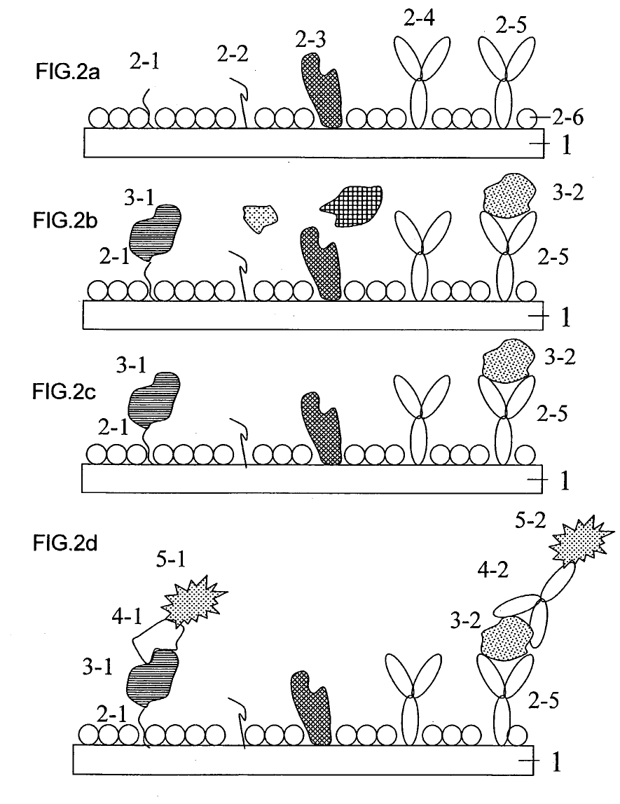

FIG. 1 is a flowchart showing the embodiment of the present invention, and FIG. 2 shows reaction models of the embodiment of the present invention.

First, a plurality of substances to be arranged, 2-1 to 2-5, such as proteins or peptides that form specific complexes with analytes, are spotted on substrate 1 such as a slide glass or membrane. Next, a masking procedure is performed for the unspotted portions. The masking procedure is performed by immersing the substrate in a masking solution (for example, when a substance that forms a specific complex with an analyte is a protein, a 0.5% skim milk, 1% BSA solution or the like may be used) for an appropriate time. 2-6 denotes the state of a masked portion (FIG. 2a). A sample containing proteins 3-1 and 3-2 (analytes) is flowed over the substrate 1, and then allowed to react for a given time, at a given temperature, to specifically bind to the substances arranged on the substrate (FIG. 2b).

After reaction, an unreacted sample is washed (FIG. 2c).

Next, substances 4-1 and 4-2 that form further specific complexes with the proteins 3-1 and 3-2 that are specifically bound to the arranged substances are labeled with fluorescence reagents or RI reagents 5-1 and 5-2. Then, the labeled substances are allowed to react on the substrate 1 for a given period of time, at a given temperature, to specifically bind to the proteins 3-1 and 3-2 on the substrate 1 (FIG. 2d).

After reaction, the unreacted labeled-substances are washed off, and then the amount of the fluorescence reagent or RI reagent excited by irradiation with excitation light is measured. Thus, the characteristics of the protein 3-1 bound to the arranged substance 2-1 and of the protein 3-2 bound to the arranged substance 2-5 can be detected, and the proteins can be identified.

In the above reaction model in FIG. 2, the characteristics of the 2 types of proteins were detected, and the proteins were identified using 5 types of the arranged substances. The present invention is not limited to this case, and encompasses all the cases wherein the characteristics of a plurality of proteins are detected and the proteins are identified using a plurality of sample substances.

### EXAMPLE

Human IL-2 monoclonal antibody, Human IL-4 monoclonal antibody and Human IL-6 monoclonal antibody were respectively adjusted with PBS (Phosphate-buffered saline (pH7.4)) to a concentration of 0.1 to 500 μg/ml, and then spotted on a PLL (Poly-L-Lysine) coated slide glass and Silylate coated glass.

After spotting, the glasses were incubated at 4° C. for approximately 10 hours. After incubation, the glasses were immersed while shaking in 1% BSA (bovine serum albumin) for approximately 1 hour to perform masking. The glasses were washed well with TPBS (0.1% Tween-20 PBS) after masking, rinsed with PBS, and then dried using a centrifugal separator. Thus the substrates were prepared and stored at 4° C. until use.

Next, the reaction of the substrate with protein analytes was performed. A 0.05% Tween-20 (v/v) and 1% BSA (w/v) PBS solution containing 1 μg/ml protein analytes (respective solutions of recombinant IL-2, IL-4, and IL-6, and a mixed solution of IL-2 and IL-6 were prepared in this time), was gently applied onto the above substrate. The substrate was covered with a cover glass, and then allowed to react at 4° C. for approximately 10 hours. After reaction, the substrate was washed well with TPBS (0.1% Tween-20 PBS), rinsed with PBS, and then dried using a centrifugal separator.

Next, the proteins were allowed to react with labeled substances. In this time, the substances had not been previously labeled. The substances specifically binding to a sample were allowed to react with the substrate, and then the substances were labeled. Mixed solutions (1 μg/ml each) of respective solutions of antibodies (biotinylated Human IL-2 monoclonal antibody, biotinylated Human IL-4 monoclonal antibody, and biotinylated Human IL-6 monoclonal antibody), which had been biotin-labeled as substances specifically binding to the samples, were adjusted with a PBS solution to have 0.05% Tween-20 (v/v) and 1% BSA (w/v), as a whole. The adjusted analytes were gently applied onto the substrate. The substrate was covered with a cover glass, and then allowed to stand at room temperature (approximately 25° C.) for 1 hour. After reaction, the substrate was washed well with TPBS (0.05% Tween-20 PBS), rinsed with PBS, and then dried using a centrifugal separator.

For labeling, Cy3 labeled streptavidin (10 μg/ml) was adjusted with a PBS solution to have 0.05% Tween-20 (v/v) and 1% BSA (w/v). The adjusted solution was gently applied over the substrate. The substrate was covered with a cover glass, and then allowed to stand at room temperature (approximately 25° C.) for 1 hour. After reaction, the substrate was washed well with TPBS (0.05% Tween-20 PBS), rinsed with PBS, and then dried using a centrifugal separator.

Subsequent to the reaction, the substrate was irradiated with excitation light. The amount of fluorescence of Cy3, specifically, the amount of fluorescence signal, was measured using a fluorescence scanner fitted with a filter with 532 nm wavelength transmission filter.

As a result, fluorescence signals corresponding to the types and concentrations of samples could be detected.

As described above, according to the present invention, the characteristics of a plurality of proteins can be simultaneously detected without labeling the proteins, which are the analytes.

Further, the kinds of proteins and to what degree they are contained in a sample, as well as their reactivities can be compared under the same conditions.

What is claimed is:

1. A protein detection method, which is a method of simultaneously detecting a plurality of proteins contained in a sample and comprises the steps of:
arranging, on a substrate, a plurality of substances to be arranged, which form specific complexes with analytes,
forming a plurality of first specific conjugates by allowing a specific binding reaction to occur on the substrate between the analytes contained in the sample and the plurality of arranged substances,
forming a plurality of second specific complexes by allowing a specific binding reaction to occur between substances, which form specific complexes with the analytes and are labeled with a fluorescence reagent or an RI reagent, and the plurality of first specific conjugates, and
identifying the analytes contained in the sample by detecting the labels contained in the plurality of second specific complexes.

2. A protein detection method, which is a method of detecting a plurality of analytes that are antigens and/or antibodies contained in a sample, and comprises the steps of:
arranging, on a substrate, a plurality of substances to be arranged, which are antibodies and/or antigens and form specific complexes with the plurality of analytes,
forming a plurality of first antigen-antibody complexes by allowing a specific antigen-antibody reaction to occur on the substrate between the analytes contained in the sample and the above plurality of arranged substances,
forming a plurality of second antigen-antibody complexes by allowing a specific antigen-antibody reaction to occur between the antibodies and/or antigens, which form specific complexes with the analytes and are labeled with a fluorescence reagent or an RI reagent, and the plurality of first antigen-antibody complexes, and
identifying the analytes contained in the sample by detecting the labeled substances contained in the second antigen-antibody complexes.

3. The protein detection method of claim 1, wherein the binding reaction that occurs during the step of forming a plurality of first specific conjugates by allowing a specific binding reaction to occur on the substrate between analytes contained in the sample and the plurality of arranged substances is selected from a specific antigen-antibody reaction between antibodies and antigens, binding reaction with peptides, interaction with proteins, enzyme reaction, and hybridization reaction with DNA; and the binding reaction that occurs during the step of forming a plurality of second specific complexes by allowing a specific binding reaction to occur between the plurality of first specific conjugates and the substances labeled with a fluorescence reagent or an RI reagent, is selected from a specific antigen-antibody reaction between antibodies and antigens, and binding reaction with peptides, interaction with proteins, enzyme reaction, and hybridization reaction with DNA.

4. The protein detection method of claim 1, which includes a step of washing samples containing analytes that remain unreacted after the binding reaction, which is between the step of forming a plurality of first specific conjugates by allowing the specific binding reaction to occur on the substrate between the analytes contained in the sample and the plurality of arranged substances, and the step of forming a plurality of second specific complexes by allowing a specific binding reaction to occur between the plurality of first specific conjugates and the substances labeled with a fluorescence reagent or an RI reagent.

5. The protein detection method of claim 2, which includes a step of washing samples containing analytes that remain unreacted after the binding reaction, which is between the step of forming a plurality of first specific conjugates by allowing the specific binding reaction to occur on the substrate between the analytes contained in the sample and the plurality of arranged substances, and the step of forming a plurality of second specific complexes by allowing a specific binding reaction to occur between the plurality of first specific conjugates and the substances labeled with a fluorescence reagent or an RI reagent.

6. The protein detection method of claim 3, which includes a step of washing samples containing analytes that remain unreacted after the binding reaction, which is between the step of forming a plurality of first specific conjugates by allowing the specific binding reaction to occur on the substrate between the analytes contained in the sample and the plurality of arranged substances, and the step of forming a plurality of second specific complexes by allowing a specific binding reaction to occur between the plurality of first specific conjugates and the substances labeled with a fluorescence reagent or an RI reagent.

7. The protein detection method of claim 1, which includes a step of removing substances that have been labeled with a fluorescence reagent or an RI reagent and remain unbound after the binding reaction, which is between the step of forming a plurality of second specific complexes by allowing the specific binding reaction to occur between the plurality of first specific conjugates and the substances labeled with the fluorescence reagent or RI reagent, and the step of identifying analytes contained in a sample by detecting the labeled substances contained in the plurality of second specific complexes.

8. The protein detection method of claim 2, which includes a step of removing substances that have been labeled with a fluorescence reagent or an RI reagent and remain unbound after the binding reaction, which is between the step of forming a plurality of second specific complexes by allowing the specific binding reaction to occur between the plurality of first specific conjugates and the substances labeled with the fluorescence reagent or RI reagent, and the step of identifying analytes contained in a sample by detecting the labeled substances contained in the plurality of second specific complexes.

9. The protein detection method of claim 3, which includes a step of removing substances that have been labeled with a fluorescence reagent or an RI reagent and remain unbound after the binding reaction, which is between the step of forming a plurality of second specific complexes by allowing the specific binding reaction to occur between the plurality of first specific conjugates and the substances labeled with the fluorescence reagent or RI reagent, and the step of identifying analytes contained in a sample by detecting the labeled substances contained in the plurality of second specific complexes.

10. The protein detection method of claim 1, which includes a step of masking portions unspotted with substances to be arranged on the substrate, which is between the step of arranging, on the substrate, a plurality of substances to be arranged, which form specific complexes with analytes, and the step of forming a plurality of first specific conjugates by allowing a specific binding reaction to occur on the substrate between the analytes contained in the sample and the plurality of arranged substances.

11. The protein detection method of claim 2, which includes a step of masking portions unspotted with substances to be arranged on the substrate, which is between the step of arranging, on the substrate, a plurality of substances to be arranged, which form specific complexes with analytes, and the step of forming a plurality of first specific conjugates by allowing a specific binding reaction to occur on the substrate between the analytes contained in the sample and the plurality of arranged substances.

12. The protein detection method of claim 3, which includes a step of masking portions unspotted with substances to be arranged on the substrate, which is between the step of arranging, on the substrate, a plurality of substances to be arranged, which form specific complexes with analytes, and the step of forming a plurality of first specific conjugates by allowing a specific binding reaction to occur on the substrate between the analytes contained in the sample and the plurality of arranged substances.
